(19) United States
(12) Patent Application Publication
(10) Pub. No.: US 2024/0321458 A1
(43) Pub. Date: Sep. 26, 2024

(54) CLINICAL CORRECTION NETWORK FOR CLINICALLY SIGNIFICANT PROSTATE CANCER PREDICTION

(71) Applicant: Siemens Healthcare GmbH, Erlangen (DE)

(72) Inventors: Andrei Puiu, Brasov (RO); Bin Lou, Princeton Junction, NJ (US); Ali Kamen, Skillman, NJ (US)

(21) Appl. No.: 18/188,494

(22) Filed: Mar. 23, 2023

(51) Int. Cl. G16H 50/30; G16H 10/60

(52) U.S. Cl. CPC G16H 50/30; G16H 10/60

(57) ABSTRACT

Systems and methods for predicting a malignancy of one or more candidate lesions are provided. One or more input medical images of a patient are received. One or more masks of one or more candidate lesions detected in the one or more input medical images are generated. A false positive reduction score of the one or more candidate lesions is determined using a machine learning based false positive reduction model based on the one or more input medical images and the one or more masks of the one or more candidate lesions. A qualification score of the one or more candidate lesions is determined using a machine learning based qualification model based on the false positive reduction score and features extracted from the one or more input medical images. A malignancy of the one or more candidate lesions is predicted using one or more machine learning based prediction networks based on the qualification score and a set of additional clinical and demographics features. The predicted malignancy of the one or more candidate lesions is output.

### TECHNICAL FIELD

The present invention relates generally to artificial intelligence/machine learning based systems for medical imaging analysis, and in particular to a clinical correction network for clinically significant prostate cancer prediction.

### BACKGROUND

Prostate cancer is one of the leading causes of cancer-related mortality among men. In the current clinical workflow, prostate cancer screening is performed by measuring the levels of PSA (prostate-specific antigen) in the blood of patients. Patients with elevated levels of PSA typically undergo a prostate biopsy to confirm prostate cancer, which is an invasive and risky procedure. Since PSA levels can fluctuate due to non-malignant factors, the specificity of PSA testing is low, leading to significant overdiagnosis of prostate cancer and overperformance of prostate biopsies.

Recently, mpMRI (multi-parametric magnetic resonance imaging) imaging has been proposed to further screen patients with elevated PSA levels, reducing the number of unnecessary prostate biopsies. However, reading an mpMRI series is a time-consuming task, requires a high level of expertise, and is highly subjective and open to interpretation. Further, radiologists typically read mpMRI series without considering clinical and demographic parameters of the patients, which carry valuable information for diagnosing prostate cancer. While CAD (computer aided diagnosis) systems have been proposed for automating the reading of an mpMRI series, current CAD systems having a high rate of false negative diagnoses of prostate cancer, resulting in insufficiently low specificity.

### BRIEF SUMMARY OF THE INVENTION

In accordance with one or more embodiments, systems and methods for improved CAD systems for, e.g., diagnosing prostate cancer are provided.

Systems and methods for predicting a malignancy of one or more candidate lesions are provided. One or more input medical images of a patient are received. One or more masks of one or more candidate lesions detected in the one or more input medical images are generated. A false positive reduction score of the one or more candidate lesions is determined using a machine learning based false positive reduction model based on the one or more input medical images and the one or more masks of the one or more candidate lesions. A qualification score of the one or more candidate lesions is determined using a machine learning based qualification model based on the false positive reduction score and features extracted from the one or more input medical images. A malignancy of the one or more candidate lesions is predicted using one or more machine learning based prediction networks based on the qualification score. The predicted malignancy of the one or more candidate lesions is output.

In one embodiment, the malignancy of the one or more candidate lesions is predicted further based on a volume of an anatomical object of interest on or in which the one or more candidate lesions are detected. In one embodiment, the malignancy of the one or more candidate lesions is predicted further based on one or more of PSA (prostate-specific antigen) of the patient, PSA density of the patient, a number of the one or more candidate lesions, or locations of the one or more candidate lesions. In one embodiment, the malignancy of the one or more candidate lesions is predicted further based on an age of the patient.

In one embodiment, the one or more masks of the one or more candidate lesions are generated by generating a mask of an anatomical object of interest depicted in the one or more input medical images and generating a mask for each of the one or more candidate lesions based on the one or more input medical images and the mask of the anatomical object using a machine learning based model.

In one embodiment, the features extracted from the one or more input medical images comprises one or more of a proportion of the one or more candidate lesions extending in a peripheral zone of a prostate of the patient, a median ADC (apparent diffusion coefficient) value of all non-candidate lesion voxels, 50th, 20th, and 10th percentiles of ADC values within each of the one or more candidate lesions, and a volume of the one or more candidate lesions.

In one embodiment, the qualification score of the one or more candidate lesions is determined further based on a detection probability of the one or more candidate lesions.

In one embodiment, the one or more input medical images comprise one or more mpMRI (multiparametric magnetic resonance imaging) images of a prostate of the patient. Predicting the malignancy of the one or more candidate lesions includes predicting prostate cancer using a prostate cancer prediction network and predicting clinically significant prostate cancer using a clinically significant prostate cancer prediction network.

These and other advantages of the invention will be apparent to those of ordinary skill in the art by reference to the following detailed description and the accompanying drawings.

### BRIEF DESCRIPTION OF THE DRAWINGS

FIG. 1 shows a method for determining a malignancy of one or more candidate lesions, in accordance with one or more embodiments;

FIG. 2 shows a workflow for automatically diagnosing prostate cancer, in accordance with one or more embodiments;

FIG. 3 shows a table of statistics of a data set used for experimentally validating embodiments described herein;

FIG. 4 shows a table comparing a conventional segmentation based detection system and a longitudinal detection network in accordance with embodiments described herein;

FIG. 5 shows a graph of Shap values for input features for evaluating embodiments described herein;

FIG. 6 shows an exemplary artificial neural network that may be used to implement one or more embodiments;

FIG. 7 shows a convolutional neural network that may be used to implement one or more embodiments; and

FIG. 8 shows a high-level block diagram of a computer that may be used to implement one or more embodiments.

### DETAILED DESCRIPTION

The present invention generally relates to methods and systems for a clinical correction network for clinically significant prostate cancer prediction. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system.

Embodiments described herein provide for improved CAD (computer aided diagnosis) systems for diagnosing prostate cancer by applying a clinical correction phase in the clinical workflow. The clinical correction phase applies a deep neural network to adjust the prediction of prostate cancer based on clinical and demographic patient data. Advantageously, by applying the clinical correction phase, the CAD systems in accordance with embodiments described herein provide for the diagnoses of prostate cancer from mpMRI images with increased specificity and performance as compared to conventional CAD systems. The CAD systems in accordance with embodiments described here generate prostate cancer diagnosis predictions that are comparable or outperform the consensus diagnoses by experienced radiologists.

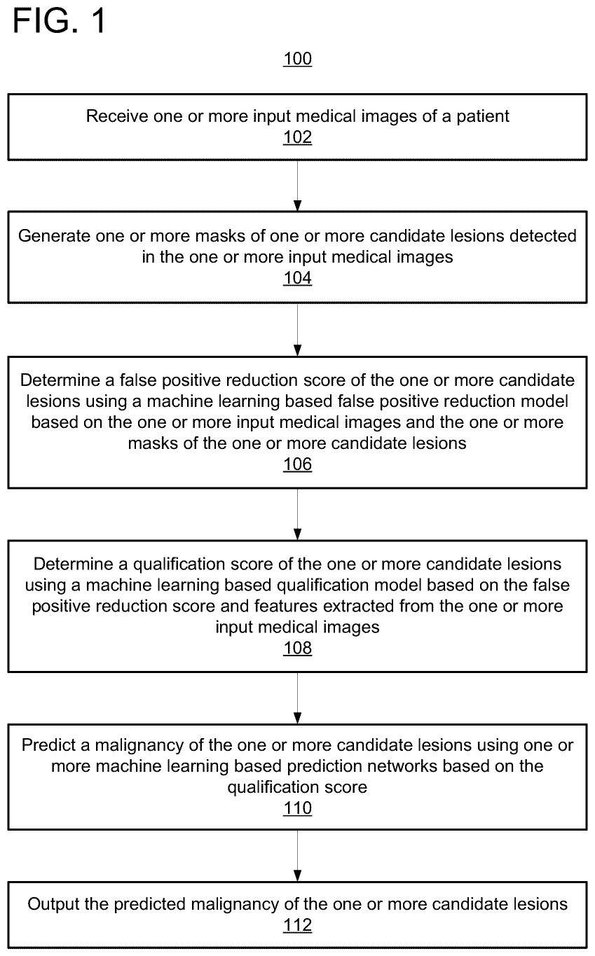

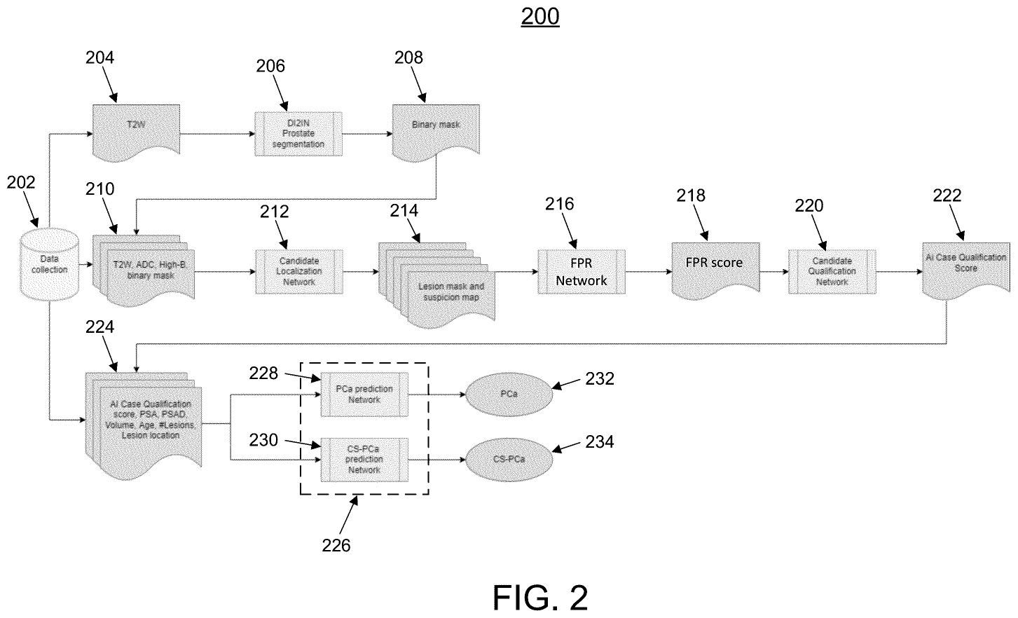

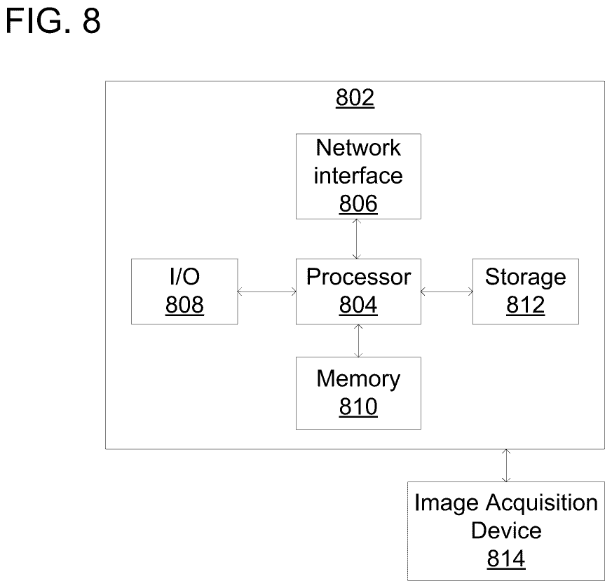

FIG. 1 shows a method 100 for determining a malignancy of one or more candidate lesions, in accordance with one or more embodiments. The steps of method 100 may be performed by one or more suitable computing devices, such as, e.g., computer 802 of FIG. 8. FIG. 2 shows a workflow 200 for automatically diagnosing prostate cancer, in accordance with one or more embodiments. FIG. 1 and FIG. 2 will be described together. The steps/operations of method 100 of FIG. 1 and workflow 200 of FIG. 2 may be implemented as part of an image acquisition device (e.g., image acquisition device 814 of FIG. 8) to provide diagnostic imaging analysis.

At step 102 of FIG. 1, one or more input medical images of a patient are received. The input medical images may depict an anatomical object of interest of the patient. In one example, the anatomical object of interest is the prostate of the patient. However, the anatomical object of interest may be an organ, a vessel, a bone, or any other suitable anatomical object.

In one embodiment, the input medical images are mpMRI medical images of the patient. For example, the mpMRI medical images may comprise T2 W (T2-weighted) images, DWI (dynamic weighted images) images, synthetic high-b DWI images (e.g., b=2000), and/or ADC (apparent diffusion coefficient) maps. The T2 W and DWI images may be extracted from DICOM (digital imaging and communications in medicine) data of the input medical images, while the synthetic high-b DWI images and the ADC maps may be computed from the DICOM data of the input medical images.

The input medical images may be of any other suitable modality, such as, e.g., other types of MRI (magnetic resonance imaging), CT (computed tomography), US (ultrasound), x-ray, or any other medical imaging modality or combinations of medical imaging modalities. The input medical images may be 2D (two dimensional) images and/or 3D (three dimensional) volumes, which may be represented as a plurality of 2D slices. Thus, reference herein to pixels of a 2D image equally refers to voxels of a 3D volume and vice versa. The input medical images may further depict different fields of view (i.e., multiscale images). The input medical images may be received, for example, directly from an image acquisition device (e.g., image acquisition device 814 of FIG. 8), such as, e.g., an MRI scanner, as the input medical images are acquired, may be received by loading previously acquired medical images from a storage or memory of a computer system, or may be received from a remote computer system. In one example, as shown in workflow 200 of FIG. 2, data collection database 202 stores the input medical images, as well as additional patient data of the patient.

At step 104 of FIG. 1, one or more masks of one or more candidate lesions detected in the one or more input medical images are generated. The one or more masks may comprise a respective mask for each of the one or more candidate lesions detected in the input medical images. The candidate lesions may comprise a candidate tumor, nodule, or any other abnormal mass in the patient. The candidate lesions are located in or on the anatomical object of interest.

In one embodiment, the masks of the candidate lesions may comprise probability maps where each respective pixel (or voxel) has an intensity value ranging from, e.g., 0 to 1 indicating a probability that the respective pixel depicts the candidate lesions. In another embodiment, additionally or alternatively, the masks may comprise binary maps generated by, for example, applying a threshold (e.g., 0.5) to the probability maps, where each respective pixel of the binary map has an intensity value of 1 indicating that the respective pixel depicts the candidate lesions or 0 indicating that the respective pixel does not depict the candidate lesions. The mask may be in any other suitable form. The masks of the candidate lesions may be generated according to any known approach.

In one embodiment, the masks of the candidate lesions are generated by first generating a mask of the anatomical object of interest depicted in the input medical images. For example, as shown in workflow 200 of FIG. 2, DI2IN (deep image-to-image network) prostate segmentation network 206 segments the prostate from T2 W images 204 (retrieved from data collection database 202). DI2IN prostate segmentation network 206 receives as input T2 W images 204 and generates as output binary mask 208 of the prostate. While prostate segmentation network 206 is shown as a DI2IN, prostate segmentation network 206 may be of any other suitable machine learning based architecture (e.g., 3D U-Net).

The masks of the candidate lesions are then generated from the input medical images based on the segmentation mask of the anatomical object of interest. For example, as shown in workflow 200 of FIG. 2, candidate localization network 212 detects candidate lesions and generates lesion masks and suspicion maps 214 of the candidate lesions. Candidate localization network 212 receives as input 210 (retrieved from data collection database 202) comprising T2 W images, ADC map, high-b DWI images and binary mask 208 and generates as output lesion binary masks and suspicion probability maps 214. In one embodiment, candidate localization network 212 is a 3D fully convolutional neural network, but may be of any other suitable machine learning based architecture.

At step 106 of FIG. 1, a false positive reduction score of the one or more candidate lesions is determined using a machine learning based false positive reduction model based on the one or more input medical images and the one or more masks of the one or more candidate lesions. The false positive reduction score represents a probability that the one of more candidate lesions detected in the input medical images are actual lesions. A higher false positive reduction score indicates a higher probability of being actual lesions and a lower probability of being false positive detections, while a lower false positive reduction score indicates a lower probability of being actual lesions and a higher probability of being false positive detections.

In one example, as shown in workflow 200 of FIG. 2, machine learning based false positive reduction model is FPR (false positive reduction) network 216. FPR network 216 receives as input lesion masks and suspicion maps 214 and input 210 comprising T2W images, ADC map, high-b DWI images and binary mask 208 and generates as output the FPR score 218. FPR network 216 may be of any suitable machine learning based architecture.

In one embodiment, a multiscale strategy is utilized to better capture the discriminative patterns. In FPR network 216, a feature encoding module is provided where three different cropped fields of view are fed into 3 groups of residual blocks independently before a fusion step. Each group comprises 3 consecutive residual blocks of CNN (convolutional neural network) architecture. After feature concatenation, an SE (squeeze-and-excitation) block is applied to allocate different weights to each channel. Another residual block is applied after the SE block for further feature fusion. The output is flattened using global average pooling and ultimately fed into 2 fully connected layers to achieve the final classification.

At step 108 of FIG. 1, a qualification score of the one or more candidate lesions is determined using a machine learning based qualification model based on the false positive reduction score and features extracted from the one or more input medical images. The qualification score also represents a probability that the one or more candidate lesions detected in the input medical images are actual lesions. A higher qualification score indicates a higher probability of being actual lesions and a lower probability of being false positive detections, while a lower qualification score indicates a lower probability of being actual lesions and a higher probability of being false positive detections.

The machine learning based false positive reduction model (utilized at step 106 of FIG. 1) is based on image patches so the false positive reduction score is only based on local image information. The machine learning based qualification model integrates information of both the localization model (e.g., candidate localization network 212) representing global information and the machine learning based false positive reduction model (e.g., FPR network 216) representing local information to generate the qualification score. The machine learning based qualification model may also use quantitative image information, such as, e.g., ADC values of lesions.

In one example, as shown in workflow 200 of FIG. 2, the machine learning based qualification model is candidate qualification network 220. Candidate qualification network 220 receives as input FPR score 218 and features extracted from the input medical images and generates as output Al case qualification score 222. The features extracted from the input medical images may comprise, for example, a proportion of candidate lesions extending in the peripheral zone of the prostate, a median ADC value of all non-candidate lesion voxels, 50th, 20th, and 10th percentile of ADC values within each candidate lesion, and the candidate lesion volume (computed from lesion masks and suspicion maps 214). In one embodiment, candidate qualification network 220 further receives as input the detection probability of the candidate lesions generated by candidate localization network 212.

Candidate qualification network 220 may be of any suitable machine learning based architecture. In one embodiment, candidate qualification network 220 comprises a fully-connected architecture with 2 hidden layers of 32 neurons each. A dropout layer with a droppage rate of 0.5 may be used between the second hidden layer and the output for regularization.

At step 110 of FIG. 1, a malignancy of the one or more candidate lesions is predicted using one or more machine learning based prediction networks based on the qualification score. The malignancy of the candidate lesions may be in any suitable form. For example, the malignancy of the candidate lesions may be a binary classification indicating that the candidate lesions are malignant or that the candidate lesions are not malignant. In another example, the malignancy of the candidate lesions may be a malignancy score representing a probability that the candidate lesions are malignant.

In one embodiment, during a clinical correction phase, the malignancy of the candidate lesions is further predicted based on patient data of the patient. In one embodiment, the patient data comprises a volume of the anatomical object of interest (e.g., the prostate gland). However, the patient information may comprise other patient clinical data (e.g., PSA, PSAD (PSA density), the number of candidate lesions, the locations of the candidate lesions, etc.) or patient demographic information (e.g., age, etc.). By incorporating such patient data, the predictive performance of the prediction networks is increased to be similar, or outperform, diagnoses by experienced radiologists.

In one example, as shown in workflow 200 of FIG. 2, the machine learning based prediction networks is clinical correction networks 226 comprising PCa (prostate cancer) prediction network 228 and CS-PCa (clinically significant prostate cancer) prediction network 230. PCa prediction network 228 and CS-PCa prediction network 230 receive as input 224 comprising Al case qualification score 222, PSA, PSAD, prostate volume, age, the number of candidate lesions, and the location of the candidate lesions. PCa prediction network 228 generates as output PCa prediction 232 and CS-PCa prediction network 226 generates as output CS-PCa prediction 234. Clinical correction networks 226 may be of any suitable machine learning based architecture. In one embodiment, clinical correction networks 226 comprises a fully-connected neural network comprising 3 hidden layers activated by hyperbolic tangent functions and an output layer with one neuron activated by a sigmoid function. Similar to candidate qualification network 220, a dropout layer is used in clinical correction networks 226 to regularize the model, stochastically cutting off 20% of the connections between the last hidden layer and the output. While PCa prediction network 228 and CS-PCa prediction network 230 are illustratively shown as separate networks in workflow 200, in some embodiments, PCa prediction network 228 and CS-PCa prediction network 230 may be implemented in a single network in a multi-task learning setting.

In one embodiment, for example where patient data is not available, the malignancy of the candidate lesions is predicted based only on the qualification score or only on the false positive reduction score. In this example, the qualification score or the false positive reduction score may be compared to a threshold. The candidate lesions are predicted to be malignant where the qualification score or the false positive reduction score satisfy (e.g., exceed) the threshold.

At step 112 of FIG. 1, the predicted malignancy of the one or more lesions is output. For example, the predicted malignancy of the one or more lesions can be output by displaying the predicted malignancy on a display device of a computer system, storing the predicted malignancy on a memory or storage of a computer system, or by transmitting the predicted malignancy to a remote computer system.

Advantageously, the CAD systems in accordance with embodiments described herein have a performance comparable to clinical PIRADS (prostate imaging reporting and data system) assigned by a consensus of experienced radiologists. By incorporating patient data, such as, e.g., volume of the anatomical object of interest (e.g., prostate gland), performance further increases to outperform the clinical PIRADS while also being completely autonomous.

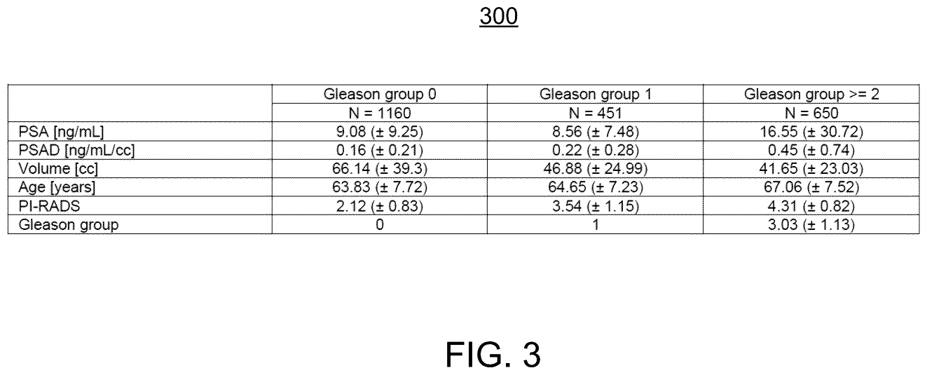

Embodiments described herein were experimentally validated using an anonymized data set comprising data from 11 clinical sites from different countries to ensure heterogeneity, leading to increased generalizability potential of the final model. The data set comprised data from 2261 patients with clinical (e.g., PSA, PSAD, etc.), demographics (e.g., age), and imaging data (mpMRI series along with PIRADS scores assigned by experienced radiologists prospectively with PSA) recorded. 48.7% of the patients were previously diagnosed with prostate cancer through a targeted biopsy as a consequence of an abnormal mpMRI (PIRADS score ≥3), out of which 59% were found to be clinically significant (Gleason group >2). Since most clinical sites do not perform systematic biopsies as part of the clinical workflow, negative cases were considered cases with a PIRADS score less than 2. All cases where the PIRADS score was above 2 and no Gleason score was available were excluded from the data set. FIG. 3 shows a table 300 of statistics of the data set used for experimentally validating embodiments described herein.

The candidate qualification network was utilized to inspect all candidate lesions identified and confirmed by the candidate localization network and the FPR network to generate a QS (qualification score) based on a series of 8 input features: detection probability, false positive reduction score, proportion of lesion extent in the peripheral zone of the prostate, median ADC values of all non-lesion voxels, 50th, 20th, and 10th percentile of ADV values within each lesion, and lesion volume computed from the suspicion probability map generated by candidate localization network. The candidate qualification network was implemented with a fully-connected architecture with 2 hidden layers of 32 neurons each. A dropout layer with a droppage rate of 0.5 has been used between the second hidden layer and the output for regularization.

During a clinical correction phase, additional patient data is utilized to further enhance system performance. A neural network is utilized to adjust the malignancy prediction based on this additional patient data. The neural network is implemented as a fully-connected neural network with 3 hidden layers activated by hyperbolic tangent functions and an output layer of one neuron activated by a sigmoid function. Similar to the candidate qualification network, a dropout layer was used to regularize the model, stochastically cutting off 20% of the connections between the last hidden layer and the output. To assess the impact of each clinical/demographic patient data, a multivariate analysis of 10 different experiments was performed: 1) QS+Age, 2) QS+PSA, 3) QS+PSAD, 4) QS+Volume (of the prostate gland), 5) QS+PSAD+Volume, 6) QS+Age+PSA, 7) QS+Age+PSAD, 8) QS+Age+Volume, 9) QS+Age+PSAD+Volume, and 10) QS+Age+PSA+PSAD+Volume.

The false positive reduction score generated by the FPR network and/or the qualification score generated by the candidate qualification network could be used per se to predict prostate cancer and/or clinically significant prostate cancer (e.g., by applying a threshold). Therefore, the system can still predict prostate cancer and/or clinically significant prostate cancer event when, for example, additional patient data is not available.

The clinical correction network was trained to minimize a BCE (binary cross entropy) loss function, as shown in Equation (1), for 300 epochs. However, a model checkpoint callback was used for better model selection, saving the best set of parameters with respect to the loss on the validation data set. Optimization was performed using the Adam optimizer with an initial learning rate of 0.0001 and a batch size of 64.

To account for class imbalance (especially in clinically significant prostate cancer predictions), class weights were defined to emphasize examples from the minority class, as reflected in Equations (1) and (2) as follows:

$$ℒ = {\sum }_{i = 0}^{{N}_{b}} {w}_{i} {y}_{i} log ( {\tilde{y}}_{l} ) + {w}_{i} ( 1 - {y}_{i} ) log ( 1 - {\tilde{y}}_{l} ) \qquad ( 1 )$$

where N_{b} is the batch size and wᵢ is the class weights defined as follows:

$${w}_{i} = \frac{N}{{\sum }_{k = 1}^{N} {δ}_{{y}_{k} {y}_{i}}} \qquad ( 2 )$$

where N is the number of samples in the training set and δ_{ab} is the Kronecker delta function:

$${δ}_{ab} = \{ \begin{matrix}1 , & if a = b \\ 0 , & if a \neq b\end{matrix} \qquad ( 3 )$$

A stratified 5-fold cross-validation scheme was used to mitigate training-validation split biases. Hence, distinct batches of 20% of the data were used iteratively as the validation data set. To increase model robustness, the final score was obtained by averaging the 5-fold scores yielded by each individual model.

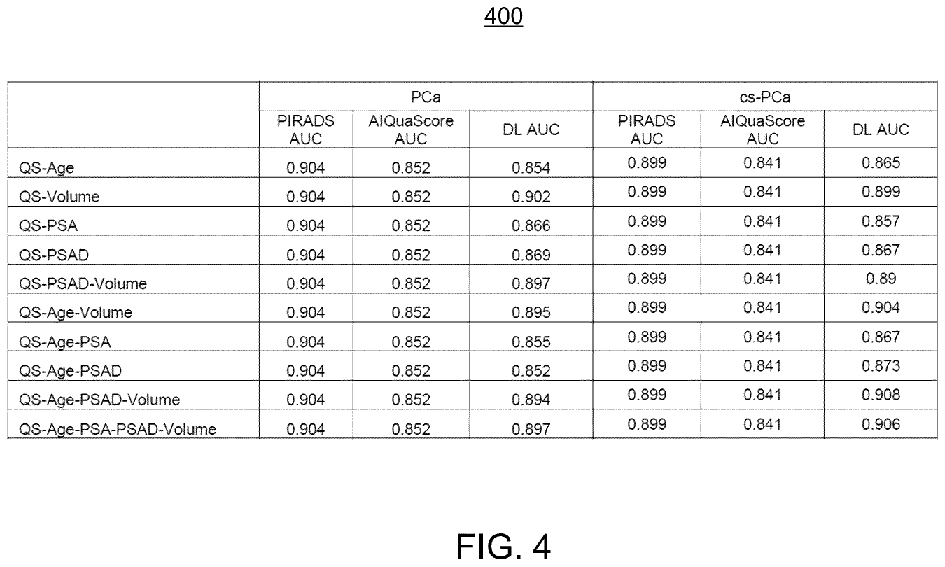

FIG. 4 shows a table 400 of results of an experimental validation of embodiments described herein. As depicted in table 400, the clinical PIRADS assigned by radiologists can predict prostate cancer with an AUC (area under curve) of 0.902 while the prostate cancer prediction based only on the qualification score only achieves an AUC of 0.852. However, the input of patient data in the clinical correction network improves the over the qualification score significantly in most of the cases, especially when the total volume of the prostate gland is taken into consideration. When only adding the volume to the qualification score as an additional input, the clinical correction network achieves an AUC of 0.902, thus reaching the performance of the radiologists (p-value=7.87 e⁻⁰¹, indicating that there is no statistically significant difference between the output of the clinical correction network and the clinical PIRADS.

For clinically significant prostate cancer, the clinical PIRADS had an AUC of 0.899 while the prediction based only on the qualification score achieves an AUC of 0.841. Consistent with the results of the prostate cancer prediction, volume information offered the largest increment in clinically significant prostate cancer prediction accuracy, improving of the qualification score approach with 5.8 points in the AUC, comparable to the radiologist performance. However, in contrast to the prostate cancer prediction, age seemed to carry additive predictive power further improving the AUC to 0.904, outperforming the clinical PIRADS. Nevertheless, PSAD used in conjunction with the qualification score, age, and gland volume further improves the AUC to 0.908, outperforming the clinical PIRADS by 0.9 points in the AUC.

To provide explainability and interpretability, a Shap analysis of the clinical correction network performance on the clinically significant prostate cancer prediction use case was performed. The Shap analysis was performed for the following input features: qualification score, prostate gland volume, age, PSAD, and PSA to compare feature value of the input features with Shap value. The Shap value represents the impact of an input feature on the output of the clinical correction network. Apart from the qualification score, the Shap analysis indicated that the most predictive feature is the total prostate gland volume, which can shift the model output by approximately +20% as follows: relatively large glands seem to reduce the risk of clinically significant prostate cancer while relatively small glands seem to increase the risk of clinically significant prostate cancer. In contrast, the patient age shows a positive correlation with the qualification score, positively shifting the prediction with age.

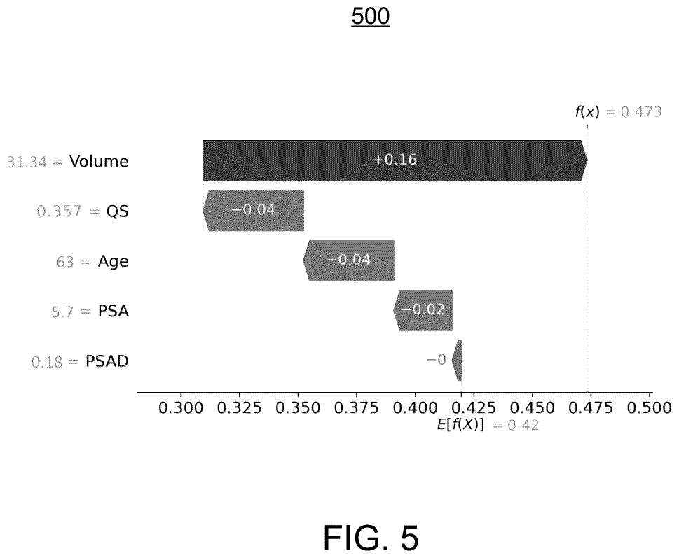

The Shap analysis was performed at the sample level as a tool to make the model prediction interpretable. FIG. 5 shows a graph 500 of Shap values for input features for evaluating embodiments described herein. As shown in graph 500, qualification score has an uncertain value (0.357), but only shifting model performed by 4%. However, the patient with a small prostate gland size (31.34 cc (cubic centimeters)), which increases the overall risk of clinically significant prostate cancer by 16%. The Shap analysis, and the visualization of graph 500, can increase trustworthiness of the model, pushing the entire system towards adoption in the clinical workflow.

Embodiments described herein are described with respect to the claimed systems as well as with respect to the claimed methods. Features, advantages or alternative embodiments herein can be assigned to the other claimed objects and vice versa. In other words, claims for the systems can be improved with features described or claimed in the context of the methods. In this case, the functional features of the method are embodied by objective units of the providing system.

Certain embodiments described herein utilize trained machine learning models, such as, e.g., the machine learning based false positive reduction model utilized at step 106, the machine learning based qualification model utilized at step 108, and the one or more machine learning based prediction networks utilized at step 110 of FIG. 1 and DI2IN prostate segmentation network 206, candidate localization network 212, FPR network 216, candidate qualification network 220, PCa prediction network 228, and CS-PCa prediction network 226 of FIG. 2. Such machine learning models are trained during a prior offline or training stage. Once trained, the trained machine learning models are utilized during an online or inference stage, for example, to perform steps or operations of method 100 of FIG. 1 and workflow 200 of FIG. 2.

Furthermore, certain embodiments described herein are described with respect to methods and systems utilizing trained machine learning based models, as well as with respect to methods and systems for training machine learning based models. Features, advantages or alternative embodiments herein can be assigned to the other claimed objects and vice versa. In other words, claims for methods and systems for training a machine learning based model can be improved with features described or claimed in context of the methods and systems for utilizing a trained machine learning based model, and vice versa.

In particular, the trained machine learning based models applied in embodiments described herein can be adapted by the methods and systems for training the machine learning based models. Furthermore, the input data of the trained machine learning based model can comprise advantageous features and embodiments of the training input data, and vice versa. Furthermore, the output data of the trained machine learning based model can comprise advantageous features and embodiments of the output training data, and vice versa.

In general, a trained machine learning based model mimics cognitive functions that humans associate with other human minds. In particular, by training based on training data, the trained machine learning based model is able to adapt to new circumstances and to detect and extrapolate patterns.

In general, parameters of a machine learning based model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the trained machine learning based model can be adapted iteratively by several steps of training.

In particular, a trained machine learning based model can comprise a neural network, a support vector machine, a decision tree, and/or a Bayesian network, and/or the trained machine learning based network can be based on k-means clustering, Q-learning, genetic algorithms, and/or association rules. In particular, a neural network can be a deep neural network, a convolutional neural network, or a convolutional deep neural network. Furthermore, a neural network can be an adversarial network, a deep adversarial network and/or a generative adversarial network.

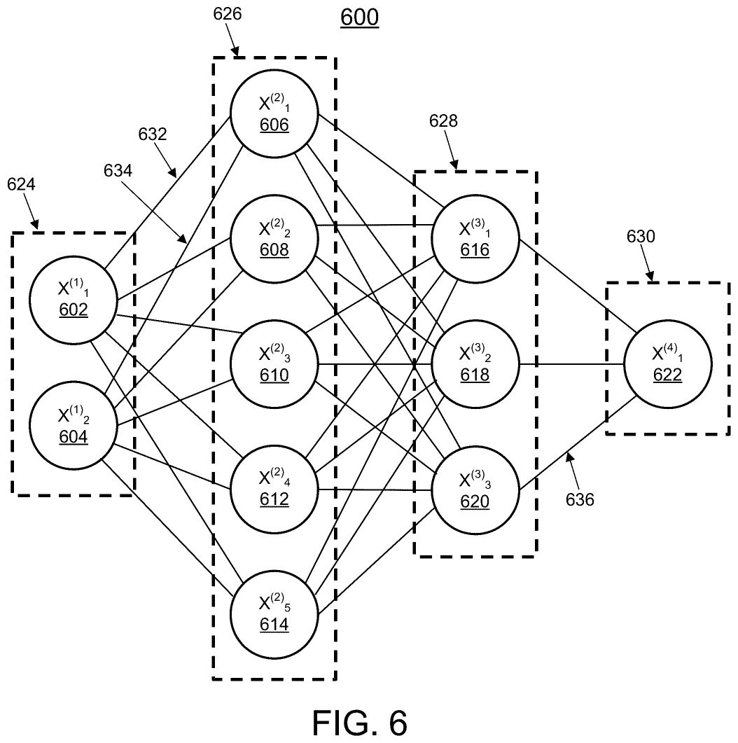

FIG. 6 shows an embodiment of an artificial neural network 600, in accordance with one or more embodiments. Alternative terms for “artificial neural network” are “neural network”, “artificial neural net” or “neural net”. Machine learning networks described herein, such as, e.g., the machine learning based false positive reduction model utilized at step 106, the machine learning based qualification model utilized at step 108, and the one or more machine learning based prediction networks utilized at step 110 of FIG. 1 and DI2IN prostate segmentation network 206, candidate localization network 212, FPR network 216, candidate qualification network 220, PCa prediction network 228, and CS-PCa prediction network 226 of FIG. 2, may be implemented using artificial neural network 600.

The artificial neural network 600 comprises nodes 602-622 and edges 632, 634, . . . , 636, wherein each edge 632, 634, . . . , 636 is a directed connection from a first node 602-622 to a second node 602-622. In general, the first node 602-622 and the second node 602-622 are different nodes 602-622, it is also possible that the first node 602-622 and the second node 602-622 are identical. For example, in FIG. 6, the edge 632 is a directed connection from the node 602 to the node 606, and the edge 634 is a directed connection from the node 604 to the node 606. An edge 632, 634, . . . , 636 from a first node 602-622 to a second node 602-622 is also denoted as “ingoing edge” for the second node 602-622 and as “outgoing edge” for the first node 602-622.

In this embodiment, the nodes 602-622 of the artificial neural network 600 can be arranged in layers 624-630, wherein the layers can comprise an intrinsic order introduced by the edges 632, 634, . . . , 636 between the nodes 602-622. In particular, edges 632, 634, . . . , 636 can exist only between neighboring layers of nodes. In the embodiment shown in FIG. 6, there is an input layer 624 comprising only nodes 602 and 604 without an incoming edge, an output layer 630 comprising only node 622 without outgoing edges, and hidden layers 626, 628 in-between the input layer 624 and the output layer 630. In general, the number of hidden layers 626, 628 can be chosen arbitrarily. The number of nodes 602 and 604 within the input layer 624 usually relates to the number of input values of the neural network 600, and the number of nodes 622 within the output layer 630 usually relates to the number of output values of the neural network 600.

In particular, a (real) number can be assigned as a value to every node 602-622 of the neural network 600. Here, x⁽ⁿ⁾ᵢ denotes the value of the i-th node 602-622 of the n-th layer 624-630. The values of the nodes 602-622 of the input layer 624 are equivalent to the input values of the neural network 600, the value of the node 622 of the output layer 630 is equivalent to the output value of the neural network 600. Furthermore, each edge 632, 634, . . . , 636 can comprise a weight being a real number, in particular, the weight is a real number within the interval [−1, 1] or within the interval [0, 1]. Here, w^{(m,n)}_{i,j} denotes the weight of the edge between the i-th node 602-622 of the m-th layer 624-630 and the j-th node 602-622 of the n-th layer 624-630. Furthermore, the abbreviation w⁽ⁿ⁾_{i,j} is defined for the weight w^{(n,n+1)}_{i,j}.

In particular, to calculate the output values of the neural network 600, the input values are propagated through the neural network. In particular, the values of the nodes 602-622 of the (n+1)-th layer 624-630 can be calculated based on the values of the nodes 602-622 of the n-th layer 624-630 by

$${x}_{j}^{( n + 1 )} = f ( {\sum }_{i} {x}_{i}^{( n )} \cdot {w}_{i , j}^{( n )} ) .$$

Herein, the function f is a transfer function (another term is “activation function”). Known transfer functions are step functions, sigmoid function (e.g. the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 624 are given by the input of the neural network 600, wherein values of the first hidden layer 626 can be calculated based on the values of the input layer 624 of the neural network, wherein values of the second hidden layer 628 can be calculated based in the values of the first hidden layer 626, etc.

In order to set the values w^{(m,n)}_{i,j} for the edges, the neural network 600 has to be trained using training data. In particular, training data comprises training input data and training output data (denoted as tᵢ). For a training step, the neural network 600 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 600 (backpropagation algorithm). In particular, the weights are changed according to

$${w}_{i , j}^{r ( n )} = {w}_{i , j}^{( n )} - γ \cdot {δ}_{j}^{( n )} \cdot {x}_{i}^{( n )}$$

wherein γ is a learning rate, and the numbers δ⁽ⁿ⁾ⱼ can be recursively calculated as

$${δ}_{j}^{( n )} = ( {\sum }_{k} {δ}_{k}^{( n + 1 )} \cdot {w}_{j , k}^{( n + 1 )} ) \cdot {f}^{′} ( {\sum }_{i} {x}_{i}^{( n )} \cdot {w}_{i , j}^{( n )} )$$

based on δ⁽ⁿ⁺¹⁾ⱼ, if the (n+1)-th layer is not the output layer, and

$${δ}_{j}^{( n )} = ( {x}_{k}^{( n + 1 )} - {t}_{j}^{( n + 1 )} ) \cdot {f}^{′} ( {\sum }_{i} {x}_{i}^{( n )} \cdot {w}_{i , j}^{( n )} )$$

if the (n+1)-th layer is the output layer 630, wherein f′ is the first derivative of the activation function, and y⁽ⁿ⁺¹⁾ⱼ is the comparison training value for the j-th node of the output layer 630.

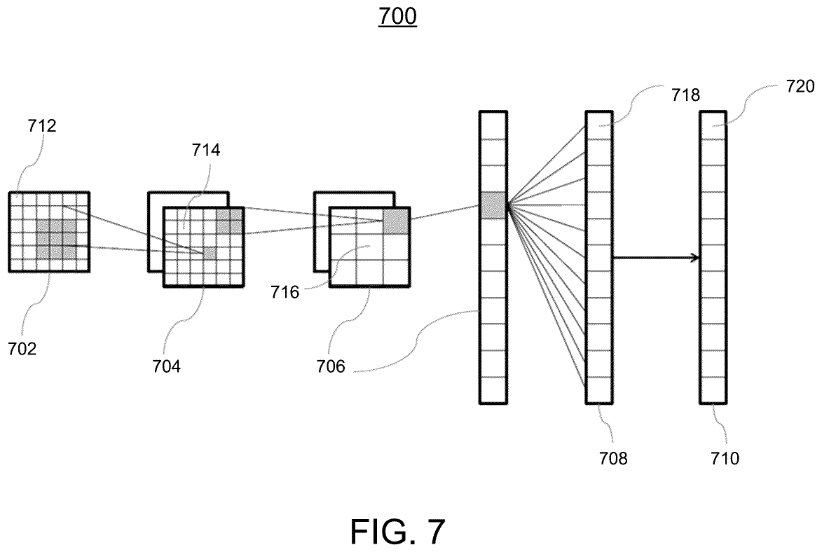

FIG. 7 shows a convolutional neural network 700, in accordance with one or more embodiments. Machine learning networks described herein, such as, e.g., the machine learning based false positive reduction model utilized at step 106, the machine learning based qualification model utilized at step 108, and the one or more machine learning based prediction networks utilized at step 110 of FIG. 1 and DI2IN prostate segmentation network 206, candidate localization network 212, FPR network 216, candidate qualification network 220, PCa prediction network 228, and CS-PCa prediction network 226 of FIG. 2, may be implemented using convolutional neural network 700.

In the embodiment shown in FIG. 7, the convolutional neural network comprises 700 an input layer 702, a convolutional layer 704, a pooling layer 706, a fully connected layer 708, and an output layer 710. Alternatively, the convolutional neural network 700 can comprise several convolutional layers 704, several pooling layers 706, and several fully connected layers 708, as well as other types of layers. The order of the layers can be chosen arbitrarily, usually fully connected layers 708 are used as the last layers before the output layer 710.

In particular, within a convolutional neural network 700, the nodes 712-720 of one layer 702-710 can be considered to be arranged as a d-dimensional matrix or as a d-dimensional image. In particular, in the two-dimensional case the value of the node 712-720 indexed with i and j in the n-th layer 702-710 can be denoted as x⁽ⁿ⁾_{[i,j]}. However, the arrangement of the nodes 712-720 of one layer 702-710 does not have an effect on the calculations executed within the convolutional neural network 700 as such, since these are given solely by the structure and the weights of the edges.

In particular, a convolutional layer 704 is characterized by the structure and the weights of the incoming edges forming a convolution operation based on a certain number of kernels. In particular, the structure and the weights of the incoming edges are chosen such that the values x⁽ⁿ⁾ₖ of the nodes 714 of the convolutional layer 704 are calculated as a convolution x⁽ⁿ⁾ₖ=Kₖ*x⁽ⁿ⁻¹⁾ based on the values x⁽ⁿ⁻¹⁾ of the nodes 712 of the preceding layer 702, where the convolution * is defined in the two-dimensional case as

$${x}_{k}^{( n )} [ i , j ] = ( {K}_{k} * {x}^{( n - 1 )} ) [ i , j ] = {\sum }_{{i}^{′}} {\sum }_{{j}^{′}} {K}_{k} [ {i}^{′} , {j}^{′} ] \cdot {x}^{( n - 1 )} [ i - {i}^{′} , j - {j}^{′} ] .$$

Here the k-th kernel Kₖ is a d-dimensional matrix (in this embodiment a two-dimensional matrix), which is usually small compared to the number of nodes 712-718 (e.g. a 3×3 matrix, or a 5×5 matrix). In particular, this implies that the weights of the incoming edges are not independent, but chosen such that they produce said convolution equation. In particular, for a kernel being a 3×3 matrix, there are only 9 independent weights (each entry of the kernel matrix corresponding to one independent weight), irrespectively of the number of nodes 712-720 in the respective layer 702-710. In particular, for a convolutional layer 704, the number of nodes 714 in the convolutional layer is equivalent to the number of nodes 712 in the preceding layer 702 multiplied with the number of kernels.

If the nodes 712 of the preceding layer 702 are arranged as a d-dimensional matrix, using a plurality of kernels can be interpreted as adding a further dimension (denoted as “depth” dimension), so that the nodes 714 of the convolutional layer 704 are arranged as a (d+1)-dimensional matrix. If the nodes 712 of the preceding layer 702 are already arranged as a (d+1)-dimensional matrix comprising a depth dimension, using a plurality of kernels can be interpreted as expanding along the depth dimension, so that the nodes 714 of the convolutional layer 704 are arranged also as a (d+1)-dimensional matrix, wherein the size of the (d+1)-dimensional matrix with respect to the depth dimension is by a factor of the number of kernels larger than in the preceding layer 702.

The advantage of using convolutional layers 704 is that spatially local correlation of the input data can exploited by enforcing a local connectivity pattern between nodes of adjacent layers, in particular by each node being connected to only a small region of the nodes of the preceding layer.

In embodiment shown in FIG. 7, the input layer 702 comprises 36 nodes 712, arranged as a two-dimensional 6×6 matrix. The convolutional layer 704 comprises 72 nodes 714, arranged as two two-dimensional 6×6 matrices, each of the two matrices being the result of a convolution of the values of the input layer with a kernel. Equivalently, the nodes 714 of the convolutional layer 704 can be interpreted as arranges as a three-dimensional 6×6×2 matrix, wherein the last dimension is the depth dimension.

A pooling layer 706 can be characterized by the structure and the weights of the incoming edges and the activation function of its nodes 716 forming a pooling operation based on a non-linear pooling function f. For example, in the two dimensional case the values x⁽ⁿ⁾ of the nodes 716 of the pooling layer 706 can be calculated based on the values x⁽ⁿ⁻¹⁾ of the nodes 714 of the preceding layer 704 as

$${x}^{( n )} [ i , j ] = f ( {x}^{( n - 1 )} [ {id}_{1} , {j}_{2} ] , … {x}^{( n - 1 )} [ {id}_{1} + {d}_{1} - 1 , {jd}_{2} + {d}_{2} - 1 ] )$$

In other words, by using a pooling layer 706, the number of nodes 714, 716 can be reduced, by replacing a number d1·d2 of neighboring nodes 714 in the preceding layer 704 with a single node 716 being calculated as a function of the values of said number of neighboring nodes in the pooling layer. In particular, the pooling function f can be the max-function, the average or the L2-Norm. In particular, for a pooling layer 706 the weights of the incoming edges are fixed and are not modified by training.

The advantage of using a pooling layer 706 is that the number of nodes 714, 716 and the number of parameters is reduced. This leads to the amount of computation in the network being reduced and to a control of overfitting.

In the embodiment shown in FIG. 7, the pooling layer 706 is a max-pooling, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

A fully-connected layer 708 can be characterized by the fact that a majority, in particular, all edges between nodes 716 of the previous layer 706 and the nodes 718 of the fully-connected layer 708 are present, and wherein the weight of each of the edges can be adjusted individually.

In this embodiment, the nodes 716 of the preceding layer 706 of the fully-connected layer 708 are displayed both as two-dimensional matrices, and additionally as non-related nodes (indicated as a line of nodes, wherein the number of nodes was reduced for a better presentability). In this embodiment, the number of nodes 718 in the fully connected layer 708 is equal to the number of nodes 716 in the preceding layer 706. Alternatively, the number of nodes 716, 718 can differ.

Furthermore, in this embodiment, the values of the nodes 720 of the output layer 710 are determined by applying the Softmax function onto the values of the nodes 718 of the preceding layer 708. By applying the Softmax function, the sum the values of all nodes 720 of the output layer 710 is 1, and all values of all nodes 720 of the output layer are real numbers between 0 and 1.

A convolutional neural network 700 can also comprise a ReLU (rectified linear units) layer or activation layers with non-linear transfer functions. In particular, the number of nodes and the structure of the nodes contained in a ReLU layer is equivalent to the number of nodes and the structure of the nodes contained in the preceding layer. In particular, the value of each node in the ReLU layer is calculated by applying a rectifying function to the value of the corresponding node of the preceding layer.

The input and output of different convolutional neural network blocks can be wired using summation (residual/dense neural networks), element-wise multiplication (attention) or other differentiable operators. Therefore, the convolutional neural network architecture can be nested rather than being sequential if the whole pipeline is differentiable.

In particular, convolutional neural networks 700 can be trained based on the backpropagation algorithm. For preventing overfitting, methods of regularization can be used, e.g. dropout of nodes 712-720, stochastic pooling, use of artificial data, weight decay based on the L1 or the L2 norm, or max norm constraints. Different loss functions can be combined for training the same neural network to reflect the joint training objectives. A subset of the neural network parameters can be excluded from optimization to retain the weights pretrained on another datasets.

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatus, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIG. 1 or 2. Certain steps or functions of the methods and workflows described herein, including one or more of the steps or functions of FIG. 1 or 2, may be performed by a server or by another processor in a network-based cloud-computing system. Certain steps or functions of the methods and workflows described herein, including one or more of the steps of FIG. 1 or 2, may be performed by a client computer in a network-based cloud computing system. The steps or functions of the methods and workflows described herein, including one or more of the steps of FIG. 1 or 2, may be performed by a server and/or by a client computer in a network-based cloud computing system, in any combination.

Systems, apparatus, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIG. 1 or 2, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 802 that may be used to implement systems, apparatus, and methods described herein is depicted in FIG. 8. Computer 802 includes a processor 804 operatively coupled to a data storage device 812 and a memory 810. Processor 804 controls the overall operation of computer 802 by executing computer program instructions that define such operations. The computer program instructions may be stored in data storage device 812, or other computer readable medium, and loaded into memory 810 when execution of the computer program instructions is desired. Thus, the method and workflow steps or functions of FIG. 1 or 2 can be defined by the computer program instructions stored in memory 810 and/or data storage device 812 and controlled by processor 804 executing the computer program instructions. For example, the computer program instructions can be implemented as computer executable code programmed by one skilled in the art to perform the method and workflow steps or functions of FIG. 1 or 2. Accordingly, by executing the computer program instructions, the processor 804 executes the method and workflow steps or functions of FIG. 1 or 2. Computer 802 may also include one or more network interfaces 806 for communicating with other devices via a network. Computer 802 may also include one or more input/output devices 808 that enable user interaction with computer 802 (e.g., display, keyboard, mouse, speakers, buttons, etc.).

Processor 804 may include both general and special purpose microprocessors, and may be the sole processor or one of multiple processors of computer 802. Processor 804 may include one or more central processing units (CPUs), for example. Processor 804, data storage device 812, and/or memory 810 may include, be supplemented by, or incorporated in, one or more application-specific integrated circuits (ASICs) and/or one or more field programmable gate arrays (FPGAs).

Data storage device 812 and memory 810 each include a tangible non-transitory computer readable storage medium. Data storage device 812, and memory 810, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 808 may include peripherals, such as a printer, scanner, display screen, etc. For example, input/output devices 808 may include a display device such as a cathode ray tube (CRT) or liquid crystal display (LCD) monitor for displaying information to the user, a keyboard, and a pointing device such as a mouse or a trackball by which the user can provide input to computer 802.

An image acquisition device 814 can be connected to the computer 802 to input image data (e.g., medical images) to the computer 802. It is possible to implement the image acquisition device 814 and the computer 802 as one device. It is also possible that the image acquisition device 814 and the computer 802 communicate wirelessly through a network. In a possible embodiment, the computer 802 can be located remotely with respect to the image acquisition device 814.

Any or all of the systems and apparatus discussed herein may be implemented using one or more computers such as computer 802.

One skilled in the art will recognize that an implementation of an actual computer or computer system may have other structures and may contain other components as well, and that FIG. 8 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A computer-implemented method comprising
receiving one or more input medical images of a patient;
generating one or more masks of one or more candidate lesions detected in the one or more input medical images;
determining a false positive reduction score of the one or more candidate lesions using a machine learning based false positive reduction model based on the one or more input medical images and the one or more masks of the one or more candidate lesions;
determining a qualification score of the one or more candidate lesions using a machine learning based qualification model based on the false positive reduction score and features extracted from the one or more input medical images;
predicting a malignancy of the one or more candidate lesions using one or more machine learning based prediction networks based on the qualification score; and
outputting the predicted malignancy of the one or more candidate lesions.

2. The computer-implemented method of claim 1, wherein predicting a malignancy of the one or more candidate lesions using one or more machine learning based prediction networks based on the qualification score comprises:
predicting the malignancy of the one or more candidate lesions further based on a volume of an anatomical object of interest on or in which the one or more candidate lesions are detected.

3. The computer-implemented method of claim 1, wherein predicting a malignancy of the one or more candidate lesions using one or more machine learning based prediction networks based on the qualification score comprises:
predicting the malignancy of the one or more candidate lesions further based on one or more of PSA (prostate-specific antigen) of the patient, PSA density of the patient, a number of the one or more candidate lesions, or locations of the one or more candidate lesions.

4. The computer-implemented method of claim 1, wherein predicting a malignancy of the one or more candidate lesions using one or more machine learning based prediction networks based on the qualification score comprises:
predicting the malignancy of the one or more candidate lesions further based on an age of the patient.

5. The computer-implemented method of claim 1, wherein generating one or more masks of one or more candidate lesions detected in the one or more input medical images comprises:
generating a mask of an anatomical object of interest depicted in the one or more input medical images; and
generating a mask for each of the one or more candidate lesions based on the one or more input medical images and the mask of the anatomical object using a machine learning based model.

6. The computer-implemented method of claim 1, wherein the features extracted from the one or more input medical images comprises one or more of a proportion of the one or more candidate lesions extending in a peripheral zone of a prostate of the patient, a median ADC (apparent diffusion coefficient) value of all non-candidate lesion voxels, 50th, 20th, and 10th percentiles of ADC values within each of the one or more candidate lesions, and a volume of the one or more candidate lesions.

7. The computer-implemented method of claim 1, wherein determining a qualification score of the one or more candidate lesions using a machine learning based qualification model based on the false positive reduction score and features extracted from the one or more input medical images comprises:
determining the qualification score of the one or more candidate lesions further based on a detection probability of the one or more candidate lesions.

8. The computer-implemented method of claim 1, wherein the one or more input medical images comprise one or more mpMRI (multiparametric magnetic resonance imaging) images of a prostate of the patient.

9. The computer-implemented method of claim 1, wherein predicting a malignancy of the one or more candidate lesions using one or more machine learning based prediction networks based on the qualification score comprises:
predicting prostate cancer using a prostate cancer prediction network; and
predicting clinically significant prostate cancer using a clinically significant prostate cancer prediction network.

10. An apparatus comprising:
means for receiving one or more input medical images of a patient;
means for generating one or more masks of one or more candidate lesions detected in the one or more input medical images;
means for determining a false positive reduction score of the one or more candidate lesions using a machine learning based false positive reduction model based on the one or more input medical images and the one or more masks of the one or more candidate lesions;
means for determining a qualification score of the one or more candidate lesions using a machine learning based qualification model based on the false positive reduction score and features extracted from the one or more input medical images;
means for predicting a malignancy of the one or more candidate lesions using one or more machine learning based prediction networks based on the qualification score; and
means for outputting the predicted malignancy of the one or more candidate lesions.

11. The apparatus of claim 10, wherein the means for predicting a malignancy of the one or more candidate lesions using one or more machine learning based prediction networks based on the qualification score comprises:
means for predicting the malignancy of the one or more candidate lesions further based on a volume of an anatomical object of interest on or in which the one or more candidate lesions are detected.

12. The apparatus of claim 10, wherein the means for predicting a malignancy of the one or more candidate lesions using one or more machine learning based prediction networks based on the qualification score comprises:
means for predicting the malignancy of the one or more candidate lesions further based on one or more of PSA (prostate-specific antigen) of the patient, PSA density of the patient, a number of the one or more candidate lesions, or locations of the one or more candidate lesions.

13. The apparatus of claim 10, wherein the means for predicting a malignancy of the one or more candidate lesions using one or more machine learning based prediction networks based on the qualification score comprises:
means for predicting the malignancy of the one or more candidate lesions further based on an age of the patient.

14. The apparatus of claim 10, wherein the means for generating one or more masks of one or more candidate lesions detected in the one or more input medical images comprises:
means for generating a mask of an anatomical object of interest depicted in the one or more input medical images; and
means for generating a mask for each of the one or more candidate lesions based on the one or more input medical images and the mask of the anatomical object using a machine learning based model.

15. A non-transitory computer readable medium storing computer program instructions, the computer program instructions when executed by a processor cause the processor to perform operations comprising:
receiving one or more input medical images of a patient;
generating one or more masks of one or more candidate lesions detected in the one or more input medical images;
determining a false positive reduction score of the one or more candidate lesions using a machine learning based false positive reduction model based on the one or more input medical images and the one or more masks of the one or more candidate lesions;
determining a qualification score of the one or more candidate lesions using a machine learning based qualification model based on the false positive reduction score and features extracted from the one or more input medical images;
predicting a malignancy of the one or more candidate lesions using one or more machine learning based prediction networks based on the qualification score; and
outputting the predicted malignancy of the one or more candidate lesions.

16. The non-transitory computer readable medium of claim 15, wherein predicting a malignancy of the one or more candidate lesions using one or more machine learning based prediction networks based on the qualification score comprises:
predicting the malignancy of the one or more candidate lesions further based on a volume of an anatomical object of interest on or in which the one or more candidate lesions are detected.

17. The non-transitory computer readable medium of claim 15, wherein the features extracted from the one or more input medical images comprises one or more of a proportion of the one or more candidate lesions extending in a peripheral zone of a prostate of the patient, a median ADC (apparent diffusion coefficient) value of all non-candidate lesion voxels, 50th, 20th, and 10th percentiles of ADC values within each of the one or more candidate lesions, and a volume of the one or more candidate lesions.

18. The non-transitory computer readable medium of claim 15, wherein determining a qualification score of the one or more candidate lesions using a machine learning based qualification model based on the false positive reduction score and features extracted from the one or more input medical images comprises:
determining the qualification score of the one or more candidate lesions further based on a detection probability of the one or more candidate lesions.

19. The non-transitory computer readable medium of claim 15, wherein the one or more input medical images comprise one or more mpMRI (multiparametric magnetic resonance imaging) images of a prostate of the patient.

20. The non-transitory computer readable medium of claim 15, wherein predicting a malignancy of the one or more candidate lesions using one or more machine learning based prediction networks based on the qualification score comprises:
predicting prostate cancer using a prostate cancer prediction network; and
predicting clinically significant prostate cancer using a clinically significant prostate cancer prediction network.
